(19) United States
(12) Patent Application Publication
(10) Pub. No.: US 2011/0229575 A1
(43) Pub. Date: Sep. 22, 2011

(54) DEEP IMMERSION FLOTATION THERAPY FOR BURN VICTIMS

(76) Inventors: Gary L. Clauson, Costa Mesa, CA (US); Chris J. Stern, Raleigh, NC (US); Richard Kiral, Costa Mesa, CA (US)

(21) Appl. No.: 13/063,348

(22) PCT Filed: Sep. 16, 2009
(86) PCT No.: PCT/US2009/005162

§ 371 Date: Jun. 3, 2011

Related U.S. Application Data
(60) Provisional application No. 61/192,536, filed on Sep. 19, 2008.

(51) Int. Cl. A61K 9/14; A61K 33/16; A61P 17/02

(52) U.S. Cl. 424/489; 424/673

(57) ABSTRACT

This invention provides compositions for and methods of treating burn wounds in a subject.

This application claims priority of U.S. Provisional Application No. 61/192,536, filed Sep. 19, 2008, the entire content of which is hereby incorporated by reference herein.

Throughout this application, various publications are referenced in parentheses. Full citations for these references may be found at the end of the specification immediately preceding the claims. The disclosures of these publications in their entireties are hereby incorporated by reference into this application to more fully describe the state of the art to which this invention pertains.

### BACKGROUND OF THE INVENTION

Burn wounds in patients are difficult to effectively treat. Tissue damage can occur in the burn wound after the initial heat and mediator damage. In addition, as well as infections, many other complications are associated with burns after the initial injury. Furthermore, burn wounds can require extended hospitalization and/or bed rest. Bed rest in turn exacerbates some problems of burn wound healing.

### SUMMARY OF THE INVENTION

A method is provided for treating a burn injury of a subject comprising immersing the subject in a liquid composition comprising an oxygenated perfluorocarbon so as to thereby treat the burn injury.

A method is provided for treating a burn injury of a subject's skin comprising immersing the skin burn injury in a liquid composition comprising an oxygenated perfluorocarbon so as to thereby treat the skin burn injury.

A method is provided for reducing skin scarring associated with a skin burn injury in a subject comprising immersing the skin burn injury in a liquid composition comprising an oxygenated perfluorocarbon so as to thereby treat the skin burn injury and thereby reduce the skin scarring.

### DETAILED DESCRIPTION OF THE INVENTION

Burn wound treatments are described in section 20, chapter 276, of The Merck Manual, 17^{th} Edition (1999), Merck Research Laboratories, Whitehouse Station, N.J., U.S.A. which is hereby incorporated by reference.

Terms

As used herein, and unless stated otherwise, each of the following terms shall have the definition set forth below.

“Burn injury” as used herein is a first, second or third degree wound caused by thermal heat, radiation, electric or chemical heat, for example as described at page 2434, section 20, chapter 276, of The Merck Manual, 17^{th} Edition (1999), Merck Research Laboratories, Whitehouse Station, N.J., U.S.A.

“Skin scarring associated with a skin burn injury” as used herein is the skin scarring response that results from a second or third degree burn.

“Mechanically circulated” as used herein means the action of moving a fluid in a closed circuit by means of a physical force.

“Filtered” as used herein means the action of passing a fluid through a physical filter, mesh or absorbent substance, so as to remove cell debris, bacteria, pathogenic organisms and/or waste products.

“Promotes alleviation of pain” as used herein means a decrease in the subject's experience of pain resulting from the burn injury.

“Accelerates healing” as used herein means an increased rate of burn injury/wound repair and healing as compared to the rate of burn injury/wound repair and healing in an untreated control subject.

Perfluorocarbons include perfluoro-tert-butylcyclohexane (C₁₀F₂₀) which is available, for example, as Oxycyte™ from Oxygen Biotherapeutics Inc., Costa Mesa, Calif. In an embodiment, the Perfluoro-tert-butylcyclohexane has the following structure:

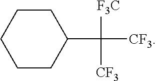

The liquid perfluorocarbon compositions may comprise pharmaceutically acceptable carrier or cosmetic carrier and adjuvant(s) suitable for topical administration. Compositions suitable for topical administration are well known in the pharmaceutical and cosmetic arts. These compositions can be adapted to comprise the oxygenated perfluorocarbon.

The compositions of the methods or uses described herein are in liquid form and are suitable for having gases bubbled through them. Non-liquid compositions that contain liquids but do not behave like liquids, such as gels, hydrogels, foams and creams and other semi-solid compositions are specifically excluded from the phrase “liquid composition” as used herein. Emulsions and other liquids are included in the phrase “liquid composition” as used herein.

An “oxygentated perfluorocarbon” as used herein is a perfluorocarbon which is carrying oxygen at, for example, saturation or sub-saturation levels.

The composition employed in the methods described herein may comprise a pharmaceutically acceptable additive.

“Topical anesthetic” means an anesthetic such as lidocaine.

“Antibacterial agent” means a bactericidal compound such as silver nitrate solution, mafenide acetate, or silver sulfadiazine, or an antibiotic.

As used herein, the term “effective” as in an amount effective to achieve and end refers to the quantity of a component that is sufficient to yield a desired therapeutic response without undue adverse side effects (such as toxicity, irritation, or allergic response) commensurate with a reasonable benefit/risk ratio when used in the manner of this disclosure. For example, an amount effective to promote burn wound healing without causing undue adverse side effects. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

It is understood that where a parameter range is provided, all integers within that range, and tenths thereof, are also provided by the invention. For example, “25-50%” includes 25.0%, 25.1%, 25.2%, 25.3%, 25.4% etc up to 50.0%. For example “10-20 mls/min” includes 10.0 mls/min, 10.1 mls/min, 10.2 mls/min, 10.3 mls/min etc. up to 20.0 mls/min.

In an embodiment of all the methods described herein the liquid composition containing a perfluorocarbon in the form of a perfluorocarbon emulsion.

In an embodiment of all the methods described herein the perfluorocarbon is perfluoro-tert-butylcyclohexane.

In an embodiment of all the methods described herein the subject is human.

In an embodiment of all the methods the perfluorocarbon is saturated with oxygen.

The perfluorocarbon emulsions of the methods and uses of the invention include perfluorocarbon-in-water emulsions comprising a continuous aqueous phase and a discontinuous perfluorocarbon phase. The emulsions can include emulsifiers, buffers, osmotic agents, and electrolytes as well as the components described hereinabove. The perfluorocarbons are present in the emulsion from about 5% to 130% w/v. Embodiments include at least about 40%, 45%, 50%, 55%, 60%, 65%, 70%, 75%, 80% and 85% w/v. A 60% w/v F-tert-butylcyclohexane emulsion may be used as the perfluorocarbon emulsion in one embodiment. Embodiments also include an egg yolk phospholipid emulsion buffered in an isotonic medium wherein the perfluorocarbon is present in the emulsion from about 5% to 130% w/v. Embodiments include at least about 40%, 45%, 50%, 55%, 60%, 65%, 70%, 75%, 80% and 85% w/v. A 60% w/v F-tert-butylcyclohexane emulsion may be used as the perfluorocarbon emulsion in one embodiment of an egg yolk phospholipid emulsion buffered in an isotonic medium.

A method is provided for treating a burn injury of a subject comprising immersing the subject in a liquid composition comprising an oxygenated perfluorocarbon so as to thereby treat the burn injury.

A method is provided for treating a burn injury of a subject's skin comprising immersing the skin burn injury in a liquid composition comprising an oxygenated perfluorocarbon so as to thereby treat the skin burn injury.

A method is provided for reducing skin scarring associated with a skin burn injury in a subject comprising immersing the skin burn injury in a liquid composition comprising an oxygenated perfluorocarbon so as to thereby treat the skin burn injury and thereby reduce the skin scarring.

In embodiments of the instant methods, the subject is partially immersed in the liquid composition. In embodiments of the instant methods, the subject is fully immersed in the liquid composition. In embodiments of the instant methods, the subject is artificially ventilated via intubation. In embodiments of the instant methods, the liquid composition accelerates healing of the burn injury. In embodiments of the instant methods, the liquid composition promotes alleviation of pain resulting from the burn injury. In embodiments of the instant methods, the liquid composition is located in a container and is mechanically circulated. In embodiments of the instant methods, liquid composition is filtered. In embodiments of the instant methods, the liquid composition is at body temperature. In embodiments of the instant methods, the liquid composition is cooled below ambient temperature. In embodiments of the instant methods, the liquid composition is heated above ambient temperature. In embodiments of the instant methods, the liquid composition is a pharmaceutical composition and comprises a pharmaceutically acceptable carrier. In embodiments of the instant methods, the liquid composition is a perflurocarbon emulsion. In embodiments of the instant methods, the perfluorocarbon emulsion has a particle size of about 0.3 microns or less. In embodiments of the instant methods, the perfluorocarbon emulsion has a particle size of about 0.05 to 0.1 microns. In embodiments of the instant methods, the liquid composition is bubbled with 1%-100% oxygen. In embodiments of the instant methods, the composition is bubbled with 100% oxygen. In embodiments of the instant methods, the composition further comprises a topical anesthetic. In embodiments of the instant methods, the composition further comprises an antibacterial agent. In embodiments of the instant methods, the perfluorocarbon is perfluoro-tert-butylcyclohexane. In embodiments of the instant methods, the subject is human.

The invention also provides use of a perfluorocarbon in the manufacture of a liquid composition for treating a skin burn injury in a subject.

The invention also provides use of a perfluorocarbon in the manufacture of a liquid composition for reducing scarring associated with skin burn injury in a subject.

In embodiments of the instant uses, the liquid composition is a pharmaceutical composition and comprises a pharmaceutically acceptable carrier. In embodiments of the instant uses, the perfluorocarbon is perfluoro-tert-butylcyclohexane. In embodiments of the instant uses, the liquid composition is manufactured to be administered at 0.1° C. to 20.0° C. below the subject's body temperature. In embodiments of the instant uses, the liquid composition is manufactured to be administered at 0.1° C. to 4.0° C. above the subject's body temperature. In embodiments of the instant uses, the subject is human.

A liquid perfluorocarbon composition is provided for use in treating as burn wound. In an embodiment, the perfluorocarbon is perfluoro-tert-butylcyclohexane.

All combinations and sub-combinations of the various elements of the methods described herein are envisaged and are within the scope of the invention.

This invention will be better understood by reference to the Experimental Details which follow, but those skilled in the art will readily appreciate that the specific experiments detailed are only illustrative of the invention which is fully set forth in the claims which follow thereafter.

Experimental Details

Disclosed herein is use of perfluorocarbon liquid as a suspending fluid and supplier of oxygen to the damaged tissues of burn victims.

Perfluorocarbons (PFCs) that are commonly used in medical research are non-toxic, biologically inert, biostatic liquids at room temperature with densities of about 1.5-2.0 g/mL and high solubilities for oxygen and carbon dioxide. Such PFCs have been found to be efficient carriers of those gases, both as emulsions for intravenous use and as neat liquids for liquid ventilation applications.

The burn victim or the burned portion of the victim, e.g. a limb, is suspended in a large container of PFC sufficient to allow the patient or portion to float freely; this removes many typical problems related to the burn patient lying in a bed with burned skin pressed against bedding.

Circulation of the perflurocarbon liquid can be employed so that the patient floats without constraints, alleviating pain associated with usual system of treatment. As the liquid is continuously circulated, external heating or cooling of the fluid can be employed in order to maintain a comfort level for the patient. The fluid is preferably filtered to assure a biologically inert and/or sterile liquid environment for the patient. Additionally, a gas mixture containing 0 to 100% oxygen from (with adequate controls and precautions for ultra-ambient levels) is bubbled in the circulating PFC to supply oxygen to the patient's skin. PFCs are excellent transporters of oxygen and carbon dioxide; being that the PFCs are slightly lipophilic at body temperature and would help in the transport of oxygen into and removal of carbon dioxide from the skin tissue, accelerating the healing process. A preferred PFC, F-tert-butylcyclohexane, is only slightly lipophilic at body temperature and not lipophilic at room temperature.

Oxycyte™ is based on the perfluorocarbon F-tert-butylcyclohexane, a saturated alicyctic PFC (molecular formula C₁₀F₂₀) and can be used as a PFC composition in the methods and uses described herein. Physical properties of F-tert-butylcyclohexane are as follows:

Molecular Formula C₁₀F₂₀

Molecular Weight (g/mol) 500.08

Physical State@Room Temp. Liquid

Density (g/mL) 1.97

Boiling Point (° C.) 147

Vapor Pressure (mmHg)@25° C. 3.8

Vapor Pressure (mmHg)@37° C. 4.4

Kinematic Viscosity (cP) 5.378

Refractive Index@20° C. 1.3098

Calculated Dipole Moment (Debye) 0.287

Calculated Surface Tension (dyne/cm) 14.4

### EXAMPLES

A subject suffering from a burn injury is floated by immersion in a composition comprising an oxygenated perfluorocarbon. The composition is regularly circulated and filtered. The subject's burn injury heals, and accelerated/improved healing is seen relative to control subject's burn injury. The subject can be suffering from a burn injury to the skin, for example a second or third degree burn.

A subject suffering from a burn injury to a limb is position so that the injured limb is immersed in a composition comprising an oxygenated perfluorocarbon. The composition is regularly circulated and filtered. The subject's burn injury heals, and accelerated/improved healing is seen relative to control subject's burn injury. The subject can be suffering from a burn injury to the skin of the limb which is a second or third degree burn.

A subject suffering from a burn injury is floated by immersion in a composition comprising an oxygenated perfluorocarbon. The composition is regularly circulated and filtered. The subject's burn injury heals, and reduced skin scarring is seen relative to scarring resulting from a control subject's burn injury.

1. A method of treating a burn injury of a subject comprising immersing the subject in a liquid composition comprising an oxygenated perfluorocarbon so as to thereby treat the burn injury.

2. A method of treating a burn injury of a subject's skin comprising immersing the skin burn injury in a liquid composition comprising an oxygenated perfluorocarbon so as to thereby treat the skin burn injury.

3. A method of reducing skin scarring associated with a skin burn injury in a subject comprising immersing the skin burn injury in a liquid composition comprising an oxygenated perfluorocarbon so as to thereby treat the skin burn injury and thereby reduce the skin scarring.

4. The method of claim 1, wherein the subject is partially immersed in the liquid composition.

5. The method of claim 1, wherein the subject is fully immersed in the liquid composition.

6. The method of claim 5, wherein the subject is artificially ventilated via intubation.

7. (canceled)

8. (canceled)

9. The method of claim 1, wherein the liquid composition is located in a container and is mechanically circulated.

10. The method of claim 9, wherein the liquid composition is filtered.

11. The method of claim 1, wherein the liquid composition is at body temperature.

12. The method of claim 1, wherein the liquid composition is cooled below ambient temperature.

13. The method of claim 1, wherein the liquid composition is heated above ambient temperature.

14. The method of claim 1, wherein the liquid composition is a pharmaceutical composition and comprises a pharmaceutically acceptable carrier.

15. The method of claim 1, wherein the liquid composition is a perflurocarbon emulsion.

16. The method of claim 15, wherein the perfluorocarbon emulsion has a particle size of about 0.3 microns or less.

17. The method of claim 15, wherein the perfluorocarbon emulsion has a particle size of about 0.05 to 0.1 microns.

18. The method of claim 1, wherein the liquid composition is bubbled with 1%-100% oxygen.

19. The method of claim 18, wherein the composition is bubbled with 100% oxygen.

20. The method of claim 1, wherein the composition further comprises a topical anesthetic.

21. The method of claim 1, wherein the composition further comprises an antibacterial agent.

22. The method of claim 1, wherein the perfluorocarbon is perfluoro-tert-butylcyclohexane.

23. The method of claim 1, wherein the subject is human.

24-32. (canceled)
